(19) United States
(12) Patent Application Publication
(10) Pub. No.: US 2011/0046046 A1
(43) Pub. Date: Feb. 24, 2011

(54) PROPHYLACTIC OR THERAPEUTIC COMPOSITION FOR DIABETES OR OBESITY

(76) Inventors: Hiroshi HARA, Sapporo-shi (JP); Tohru HIRA, Sapporo-shi (JP); Yuzuro ETO, Kawasaki-shi (JP)

Correspondence Address: CERMAK NAKAJIMA LLP;ACS LLC, 127 S. Peyton Street, Suite 210, ALEXANDRIA, VA, 22314, US

(21) Appl. No.: 12/861,929

(22) Filed: Aug. 24, 2010

Related U.S. Application Data
(63) Continuation of application No. PCT/JP2009/053409, filed on Feb. 25, 2009.

(30) Foreign Application Priority Data
Feb. 25, 2008 (JP) 2008-043348

(51) Int. Cl. A61K 38/06; A61K 8/41; A61K 38/05; A61P 3/04; A61P 3/10

(52) U.S. Cl. 514/4.8; 514/654; 514/6.9

(57) ABSTRACT

Disclosed is a safe and non-toxic prophylactic or therapeutic composition for diabetes or obesity. The prophylactic or therapeutic composition for diabetes or obesity comprises as an active ingredient, a calcium receptor activator such as γ-Glu-X-Gly, wherein X represents an amino acid or an amino acid derivative; γ-Glu-Val-Y, wherein Y represents an amino acid or an amino acid derivative; γ-Glu-Ala; γ-Glu-Gly; γ-Glu-Cys; γ-Glu-Met; γ-Glu-Thr; γ-Glu-Val; γ-Glu-Orn; Asp-Gly; Cys-Gly; Cys-Met; Glu-Cys; Gly-Cys; Leu-Asp; γ-Glu-Met(O); γ-Glu-γ-Glu-Val; γ-Glu-Val-NH₂; γ-Glu-Val-ol; γ-Glu-Ser; γ-Glu-Tau; γ-Glu-Cys(S-Me)(O); γ-Glu-Leu; γ-Glu-Ile; γ-Glu-t-Leu; γ-Glu-Cys(S-Me); cinacalcet; a cinacalcet analogue compound and protamine.

This application is a continuation under 35 U.S.C. §120 of PCT Patent Application No. PCT/JP2009/053409, filed Feb. 25, 2009, which claims priority under 35 U.S.C. §119 to Japanese Patent Application No. 2008-043348, filed on Feb. 25, 2008, which are incorporated in their entireties by reference.

### BACKGROUND OF THE INVENTION

1. Field of the Invention

The present invention relates to a prophylactic or therapeutic composition for diabetes or obesity, which includes a peptide or the like. The peptide is a calcium receptor activator, and is present in the composition as the active ingredient.

2. Brief Description of the Related Art

In modern society, obesity, hypertension, impaired glucose tolerance, and dyslipidemia have been identified as the four major diseases owing to the increased prevalence of a high-fat and high-calorie diet. As a result, the incidence of heart disease or arteriosclerosis increases, which puts our lives at risk. Therefore, there is a demand for the development of an effective method for prophylaxis or treatment of diabetes or obesity.

In vivo energy metabolism is controlled by insulin which is produced in pancreatic β cells. Insulin acts on peripheral tissues and cells and promotes the intake of sugar from blood, thereby playing an important role in the control of the blood-sugar level. When the insulin sensitivity of cells is lowered by the continuous intake of a high-calorie diet, the elevation of the blood-sugar level is proportionate to the excess secretion of insulin. As a result, the pancreatic β cells become exhausted and dysfunctional, leading to the development of diabetes and obesity.

The secretion of insulin is regulated by various hormones, and in particular, a glucagon-like peptide-1 (GLP-1), which is produced and secreted in the gastrointestinal tract, and is considered to be important in insulin regulation. GLP-1 is a peptide hormone having a molecular weight of about 4000, and is mainly produced in the L cells of the small intestine. It has been reported that GLP-1 has a function, for example, of promoting the secretion of insulin from β cells, suppressing gastric emptying, suppressing appetite or overeating, and is also effective for treatment and prophylaxis of diabetes and obesity. It is known that a decline in the ability to produce GLP-1 is observed in diabetes and obesity. Thus, the promotion of the production of GLP-1 in those conditions is expected to lead to the treatment and prophylaxis of diabetes and obesity. The production of GLP-1 in L cells is promoted by the intake of various nutrients such as carbohydrates, lipids, and proteins, but is rare where a compound such as a peptide of a specific ingredient is employed as a GLP-1 secretion promoter.

Cholecystokinin (CCK) is a peptide hormone having a molecular weight of about 1000-4000. CCK is mainly produced in the I cells of the duodenum and the small intestine, and promotes the secretion of bile and pancreatic digestive juice. CCK has many physiological functions, some of which include suppressing the gastric emptying of foods, promoting the secretion of pancreatic enzymes, and suppressing food intake through a sensation of fullness (Science, vol. 247, p. 1589-1591, 1990 and American Journal of Physiology, vol. 276, R1701-R1709, 1999). Additionally, CCK functions to promote the secretion of insulin as a blood sugar-regulating hormone (Diabetes, vol. 36, p. 1212-1215, 1987 and Journal of Clinical Endocrinological Metabolism, vol. 65, p. 395-401, 1987). With such functions, CCK is considered to be promising for treatment or prophylaxis of lifestyle-related diseases such as diabetes, obesity, and pancreatitis.

GLP-1 and CCK are both peptide hormones. Thus, in a therapeutic application, GLP-1 and CCK are administered into the blood by injection or a similar means, which is not ideal due to the complications of daily administration and the significant related expenses. However, it is conceivable to use such a mechanism where endogenous GLP-1 and CCK are secreted from GLP-1- and CCK-producing cells present in the small intestinal mucosa by using proteins, peptides, amino acids, fatty acids, and the like, as food ingredients. In other words, there has been a demand for the development of a compound or a food material having a function of promoting the secretion of GLP-1 and CCK.

A calcium receptor, known as Calcium Sensing Receptor (CaSR), is formed of 1078 amino acids, and is classified into the class C seven-transmembrane receptors (G protein-coupled receptor). Cloning of the gene for the calcium receptor was reported in 1993 (Nature, 1993, vol. 366 (6455), p. 575-580), and the calcium receptor is known to cause various cell responses through the elevation of the intracellular calcium level or the like, when being activated with calcium or the like. The gene sequence of the human calcium receptor is registered with GenBank Accession No. NM_{—}000388 and is well conserved in animals.

In addition to calcium, many other calcium receptor activators have been reported, including but not limited to, cations such as a gadolinium cation, basic peptides such as polyarginine, polyamines such as spermine, proteins such as protamine, amino acids such as phenylalanine, and so forth (Cell Calcium, 2004, vol. 35 (3), p. 209-216).

The article, J. Biol. Chem., 2006, Vol. 281 (13), p. 8864-8870, reports that glutathione (γ-Glu-Cys-Gly), as a low molecular weight peptide, is a calcium receptor activator (that is, it has an activating function), but does not disclose that glutathione can be effective for prophylaxis or treatment of diabetes and obesity.

Similarly, WO 2007/055388 A1, which discloses that a dipeptide or a tripeptide having a specific sequence is effective as a calcium receptor activator, also describes a possibility for the dipeptide or the tripeptide to be used as a therapeutic drug for various diseases, but does not disclose that the dipeptide or the tripeptide is effective for diabetes and obesity.

### SUMMARY OF THE INVENTION

As described above, lifestyle-related diseases, in particular, diabetes and obesity have become a serious social issue. For the prophylaxis or treatment of those lifestyle-related diseases, there is a need for the development of pharmaceuticals, foods or useful substances equivalent to the foods, which are safe and non-toxic for humans and animals. Therefore, an aspect of the present invention is to provide a prophylactic or therapeutic composition for diabetes, obesity, or both, which promotes the production of GLP-1, CCK, or both in a gastrointestinal tract tissue, and which is safe and non-toxic for humans and animals.

It has been found that a calcium receptor activator functions to promote the secretion of GLP-1 and CCK from GLUTag and STC-1 cells derived from the intestinal tract. Specifically, it has been found that a peptide or a low molecular weight compound having a calcium receptor-activating function, as described in the examples mentioned below, promotes the secretion of GLP-1 and CCK, and thus, can serve as a prophylactic or therapeutic composition indicated for diabetes and obesity.

It is an aspect of the present invention to provide a prophylactic or therapeutic composition for diabetes or obesity, comprising a calcium receptor activator.

It is a further aspect of the present invention to provide the prophylactic or therapeutic composition for diabetes or obesity as described above, wherein the calcium receptor activator is selected from the group consisting of γ-Glu-X-Gly, where X represents an amino acid or an amino acid derivative; γ-Glu-Val-Y, where Y represents an amino acid or an amino acid derivative; γ-Glu-Ala; γ-Glu-Gly; γ-Glu-Cys; γ-Glu-Met; γ-Glu-Thr; γ-Glu-Val; γ-Glu-Orn; Asp-Gly; Cys-Gly; Cys-Met; Glu-Cys; Gly-Cys; Leu-Asp; γ-Glu-Met(O); γ-Glu-γ-Glu-Val; γ-Glu-Val-NH₂; γ-Glu-Val-ol; γ-Glu-Ser; γ-Glu-Tau; γ-Glu-Cys(S-Me)(O); γ-Glu-Leu; γ-Glu-t-Leu; γ-Glu-Cys(S-Me); cinacalcet; an analogous compound of cinacalcet; and combinations thereof.

It is a further aspect of the present invention to provide the prophylactic or therapeutic composition for diabetes or obesity as described above, wherein X is selected from the group consisting of Cys(SNO), Cys(S-allyl), Gly, Cys(S-Me), Abu, and Ser; and Y is selected from the group consisting of Gly, Val, Glu, Lys, Phe, Ser, Pro, Arg, Asp, Met, Thr, His, Orn, Asn, Cys, and Gln.

It is a further aspect of the present invention to provide the prophylactic or therapeutic composition for diabetes or obesity as described above, wherein the calcium receptor activator is selected from the group consisting of γ-Glu-Val-Gly, γ-Glu-Cys-Gly, and cinacalcet.

It is a further aspect of the present invention to provide the prophylactic or therapeutic composition for diabetes or obesity as described above, wherein the calcium receptor activator is γ-Glu-Cys.

It is a further aspect of the present invention to provide the prophylactic or therapeutic composition for diabetes or obesity as described above, wherein the calcium receptor activator is protamine.

It is a further aspect of the present invention to provide a food for prophylaxis or treatment of diabetes or obesity, comprising γ-Glu-Cys-Gly in an amount of 0.000001% or more.

It is a further aspect of the present invention to provide a food for prophylaxis or treatment of diabetes or obesity as described above, comprising γ-Glu-Cys in an amount of 0.000001% or more.

It is a further aspect of the present invention to provide a food for prophylaxis or treatment of diabetes or obesity as described above, comprising protamine in an amount of 0.000001% or more.

It is a further aspect of the present invention to provide a use of a calcium receptor activator for production of a prophylactic or therapeutic composition for diabetes or obesity.

It is a further aspect of the present invention to provide the use as described above, wherein the calcium receptor activator is selected from the group consisting of γ-Glu-X-Gly, where X represents an amino acid or an amino acid derivative; γ-Glu-Val-Y, where Y represents an amino acid or an amino acid derivative; γ-Glu-Ala; γ-Glu-Gly; γ-Glu-Cys; γ-Glu-Met; γ-Glu-Thr; γ-Glu-Val; γ-Glu-Orn; Asp-Gly; Cys-Gly; Cys-Met; Glu-Cys; Gly-Cys; Leu-Asp; γ-Glu-Met(O); γ-Glu-Val-NH₂; γ-Glu-Val-ol; γ-Glu-Ser; γ-Glu-Tau; γ-Glu-Cys(S-Me)(O); γ-Glu-Leu; γ-Glu-t-Leu; γ-Glu-Cys(S-Me); cinacalcet; and an analogous compound of cinacalcet.

It is a further aspect of the present invention to provide the use as described above, wherein X represents Cys(SNO), Cys(S-allyl), Gly, Cys(S-Me), Abu, or Ser, and Y represents Gly, Val, Glu, Lys, Phe, Ser, Pro, Arg, Asp, Met, Thr, His, Orn, Asn, Cys, or Gln.

It is a further aspect of the present invention to provide the use as described above, wherein the calcium receptor activator is γ-Glu-Val-Gly, γ-Glu-Cys-Gly, or cinacalcet.

It is a further aspect of the present invention to provide the use as described above, wherein the calcium receptor activator is γ-Glu-Cys.

It is a further aspect of the present invention to provide the use as described above, wherein the calcium receptor activator is protamine.

It is a further aspect of the present invention to provide a method for prophylaxis or treatment of diabetes or obesity, comprising administering a composition comprising a calcium receptor activator to a subject in need of prophylaxis or treatment of diabetes or obesity.

It is a further aspect of the present invention to provide the method as described above, wherein the calcium receptor activator is selected from the group consisting of γ-Glu-X-Gly, where X represents an amino acid or an amino acid derivative except for cysteine; γ-Glu-Val-Y, where Y represents an amino acid or an amino acid derivative; γ-Glu-Ala; γ-Glu-Gly; γ-Glu-Cys; γ-Glu-Met; γ-Glu-Thr; γ-Glu-Val; γ-Glu-Orn; Asp-Gly; Cys-Gly; Cys-Met; Glu-Cys; Gly-Cys; Leu-Asp; γ-Glu-Met(O); γ-Glu-γ-Glu-Val; γ-Glu-Val-NH₂; γ-Glu-Val-ol; γ-Glu-Ser; γ-Glu-Tau; γ-Glu-Cys(S-Me)(O); γ-Glu-Leu; γ-Glu-t-Leu; γ-Glu-Cys(S-Me); cinacalcet; an analogous compound of cinacalcet; and combinations thereof.

It is a further aspect of the present invention to provide the method as described above, wherein X is selected from the group consisting of Cys(SNO), Cys(S-allyl), Gly, Cys(S-Me), Abu, and Ser; and Y is selected from the group consisting of Gly, Val, Glu, Lys, Phe, Ser, Pro, Arg, Asp, Met, Thr, His, Orn, Asn, Cys, and Gln.

It is a further aspect of the present invention to provide the method as described above, wherein the calcium receptor activator is γ-Glu-Val-Gly or cinacalcet.

It is a further aspect of the present invention to provide the method as described above, wherein the calcium receptor activator is γ-Glu-Cys.

It is a further aspect of the present invention to provide the method as described above, wherein the calcium receptor activator is protamine.

It is a further aspect of the present invention to provide the method as described above, wherein the composition comprises γ-Glu-Cys in an amount of 0.000001% or more.

It is a further aspect of the present invention to provide the method as described above, wherein the composition comprises protamine in an amount of 0.000001% or more.

### BRIEF DESCRIPTION OF THE DRAWINGS

FIG. 1 shows a series of graphs illustrating the promotion of secretion of CCK from STC-1 cells by γ-Glu-Cys-Gly, γ-Glu-Val-Gly, and cinacalcet. In the graphs, Blk, γEVG, GSH, and CCT represent a blank (control), γ-Glu-Val-Gly, γ-Glu-Cys-Gly, and cinacalcet, respectively. The concentrations represent final concentrations in wells. The same is applied in FIGS. 2 and 3.

FIG. 2 shows a graph illustrating the promotion of secretion of GLP-1 from GLUTag cells by γ-Glu-Cys-Gly and cinacalcet.

FIG. 3 shows a graph illustrating the promotion of secretion of GLP-1 from GLUTag cells by γ-Glu-Cys and protamine. In the graph, γEC represents γ-Glu-Cys.

FIG. 4 shows a graph illustrating a change in GLP-1 secretion from the rat intestinal tract by γ-Glu-Cys (EC). The values are plotted as average values with standard errors (n=8). The symbol “+” means that there is a statistically significant difference with respect to 0 minute, and the symbol “*” means that there is a statistically significant difference with respect to a water administration group (Tukey's test, P<0.05).

FIG. 5 shows a graph illustrating an action of suppressing the elevation of blood sugar by γ-Glu-Cys (EC) in a rat. The values are plotted as average values with standard errors (n=8). The symbol “*” means that there is a statistically significant difference with respect to a glucose solution administration group (Tukey's test, P<0.05).

### DETAILED DESCRIPTION OF EXEMPLARY EMBODIMENTS

A prophylactic or therapeutic composition for diabetes or obesity can include a calcium receptor activator as an active ingredient. The calcium receptor activator can be a peptide or a low molecular weight compound having a calcium receptor-activating function.

The term “calcium receptor” can refer to a receptor that is known as Calcium Sensing Receptor (CaSR) and belongs to the class C seven-transmembrane receptors. The term “calcium receptor activator” can also refer to a substance that binds to the above-mentioned calcium receptor to activate the calcium receptor and control functions of cells which express the calcium receptor. Furthermore, the phrase “to activate a calcium receptor” can mean that a ligand binds to a calcium receptor to activate a guanine nucleotide binding protein, to thereby transmit a signal. Additionally, the term “calcium receptor activity” can also mean that the calcium receptor transmits the signal.

<1>Peptide or Low Molecular Weight Compound Having a Calcium Receptor-Activating Function

The peptide or the low molecular weight compound having a calcium receptor-activating function may be obtained, for example, by reacting a calcium receptor and a test substance with each other, and detecting any calcium receptor activity. It can then be confirmed whether the obtained peptide or low molecular weight compound functions to promote the secretion of GLP-1 or CCK, or has a prophylactic or therapeutic effect on diabetes or obesity.

Hereinafter, a method of screening the peptide or the low molecular weight compound having a calcium receptor-activating function is specifically described, but is not limited to these steps including:

1) measuring a calcium receptor activity by adding a test substance to a calcium receptor activity measurement system for measuring the calcium receptor activity;

2) comparing a calcium receptor activity with the test substance to a calcium receptor activity without the test substance; and

3) selecting the test substance exhibiting a high calcium receptor activity with the test substance.

The calcium receptor activity is measured, for example, by using a measurement system using cells that express calcium receptors. These cells may be endogenously expressing calcium receptors, or recombinant cells wherein exogenous calcium receptor genes are introduced. The above-mentioned calcium receptor activity measurement system may be used without any particular limitation as long as, when an extracellular ligand (activator) specific to a calcium receptor is added to the above-mentioned cells that express calcium receptors, the measurement system can detect the binding (reaction) between the activator and the calcium receptor, or can respond to the binding (reaction) between the activator and the calcium receptor to thereby transmit a detectable signal into the cells. When the calcium receptor activity is detected through the reaction with the test substance, it is confirmed that the test substance has a calcium receptor-stimulating activity, and is a substance having a prophylactic or therapeutic effect on diabetes or obesity.

Furthermore, the prophylactic or therapeutic effect on diabetes or obesity, which the calcium receptor activator possesses, may be examined, for example, by such a test for examining a function of promoting the secretion of GLP-1 or CCK as described in the examples below. Moreover, the prophylactic or therapeutic effect on diabetes may also be confirmed, for example, by such a glucose tolerance test for examining an effect of suppressing the elevation of blood sugar as described in the examples below. Additionally, the peptide and the low molecular weight compound to be used as a test substance is not particularly limited, and the peptide can be a peptide formed of 2 to 10 amino acid residues or a derivative thereof, or a peptide formed of 2 or 3 amino acid residues or a derivative thereof. Furthermore, the amino acid residue at the N-terminal side of the peptide can be γ-glutamic acid. The low molecular weight compound can be cinacalcet ((R)-N-(3-(3-(trifluoromethyl)phenyl)propyl)-1-(1-naphthyl)ethylamine) or an analogous compound thereof. The analogous compound of cinacalcet is described later.

The origin of the above-mentioned calcium receptor is not particularly limited. For example, the origins include, but are not limited to, the above-mentioned human calcium receptor, and a calcium receptor derived from an animal such as a mouse, a rat, and a dog. Specifically, examples of the calcium receptor may include the human calcium receptor, encoded by the human calcium receptor gene, registered with GenBank Accession No NM_{—}00388. The calcium receptor is not limited to the protein encoded by the gene having the above-mentioned sequence, and may be a protein encoded by a gene having a 60% or more, or in another example 80% or more, and in yet another example 90% or more homology to the above-mentioned sequence, as long as the gene encodes a protein having a calcium receptor function. The GPRC6A receptor, or 5.24 receptor, is also known as a subtype of the calcium receptor, and may be used. The calcium receptor function may be investigated by monitoring the expression of those genes in cells, and measuring the change in current at the time of the addition of calcium, and the change in the intracellular calcium ion concentration.

As described above, the calcium receptor activity may be confirmed by using live cells expressing a calcium receptor or its fragment, cell membranes expressing a calcium receptor or its fragment, an in vitro system containing a protein of a calcium receptor or its fragment, or the like.

An example using live cells is described below. However, confirmation of the calcium receptor activity is not limited to this example.

A calcium receptor is expressed in cultured cells such as Xenopus laevis oocytes, hamster ovarian cells, and human fetal kidney cells. The calcium receptor can be expressed by cloning a calcium receptor gene in a plasmid that carries a foreign gene, and introducing the plasmid or cRNA obtained by using the plasmid as a template. To detect the reaction, an electrophysiological technique and a fluorescent indicator that indicates an increase in intracellular calcium level may be used.

Expression of the calcium receptor is first confirmed based on the response to calcium or a specific activator. Oocytes showing intracellular current with calcium at a concentration of about 5 mM, or cultured cells showing fluorescence of the fluorescent indicator reagent with calcium at a concentration of about 5 mM can be used. The calcium concentration dependency can be determined by changing the calcium concentration. Then, a test substance such as a peptide is added to the oocytes or cultured cells to a concentration of about 1 μM to 1 mM, and the calcium receptor activity of the above-mentioned peptide or the like is determined.

Examples of peptides having a calcium receptor-activating activity include γ-Glu-X-Gly, where X represents an amino acid or an amino acid derivative, γ-Glu-Val-Y, where Y represents an amino acid or an amino acid derivative, γ-Glu-Ala, γ-Glu-Gly, γ-Glu-Cys, γ-Glu-Met, γ-Glu-Thr, γ-Glu-Val, γ-Glu-Orn, Asp-Gly, Cys-Gly, Cys-Met, Glu-Cys, Gly-Cys, Leu-Asp, γ-Glu-Met(O), γ-Glu-γ-Glu-Val, γ-Glu-Val-NH₂, γ-Glu-Val-ol, γ-Glu-Ser, γ-Glu-Tau, γ-Glu-Cys(S-Me)(O), γ-Glu-Leu, γ-Glu-t-Leu, and γ-Glu-Cys(S-Me) (hereinafter, also referred to as a “peptide of the present invention”).

Of the above-mentioned compounds, examples include those wherein X represents Cys, Cys(SNO), Cys(S-allyl), Gly, Cys(S-Me), Abu, or Ser, and Y represents Gly, Val, Glu, Lys, Phe, Ser, Pro, Arg, Asp, Met, Thr, His, Orn, Asn, Cys, or Gln.

It should be noted that an amino acid of which each peptide is formed is an L-amino acid unless otherwise stated. Herein, examples of the amino acid include, but are not limited to: a neutral amino acid such as Gly, Ala, Val, Leu, Ile, Ser, Thr, Cys, Met, Asn, Gln, Pro, and Hyp; an acidic amino acid such as Asp and Glu; a basic amino acid such as Lys, Arg, and His; an aromatic amino acid such as Phe, Tyr, and Trp; and homoserine, citrulline, ornithine, α-aminobutyric acid, norvaline, norleucine, and taurine.

In this description, abbreviations for amino group residues can represent the following amino acids.

| Amino Group | |
|---|---|
| Residue | |
| Abbreviations | Amino Acids |
| (1) Gly | Glycine |
| (2) Ala | Alanine |
| (3) Val | Valine |
| (4) Leu | Leucine |
| (5) Ile | Isoleucine |
| (6) Met | Methionine |
| (7) Phe | Phenylalanine |
| (8) Tyr | Tyrosine |
| (9) Trp | Tryptophan |
| (10) His | Histidine |
| (11) Lys | Lysine |
| (12) Arg | Arginine |
| (13) Ser | Serine |
| (14) Thr | Threonine |
| (15) Asp | Aspartic acid |
| (16) Glu | Glutamic acid |
| (17) Asn | Asparagine |
| (18) Gln | Glutamine |
| (19) Cys | Cysteine |
| (20) Pro | Proline |
| (21) Orn | Ornithine |
| (22) Sar | Sarcosine |
| (23) Cit | Citrulline |
| (24) N-Val | Norvaline |
| (25) N-Leu | Norleucine |
| (26) Abu | α-Aminobutyric |
| | acid |
| (27) Tau | Taurine |
| (28) Hyp | Hydroxyproline |
| (29) t-Leu | tert-Leucine |

Furthermore, examples of the amino acid derivative include various derivatives of the above-mentioned amino acids such as an unusual amino acid, a non-natural amino acid, an amino alcohol, and a substituted amino acid, where an amino acid side chain such as the terminal carbonyl group, the terminal amino group, and the thiol group of cysteine, are substituted with various substituents. Examples of the substituents include, but are not limited to, an alkyl group, an acyl group, a hydroxy group, an amino group, an alkylamino group, a nitro group, a sulfonyl group, and various protection groups. Examples of the substituted amino acid include Arg(NO₂): N-γ-nitroarginine; Cys(SNO): S-nitrocysteine; Cys(S-Me): S-methylcysteine; Cys(S-allyl): S-allylcysteine; Val-NH₂: valinamide; and Val-ol: valinol (2-amino-3-methyl-1-butanol).

It should be noted that γ-Glu-Cys(SNO)-Gly described above has the following structural formula indicated below, and the “(O)” in the above-mentioned formulae γ-Glu-Met(O) and γ-Glu-Cys(S-Me)(O) indicates a sulfoxide structure. The “γ” in γ-Glu indicates that the glutamic acid binds to another amino acid via the carboxy group at the y position of the glutamic acid.

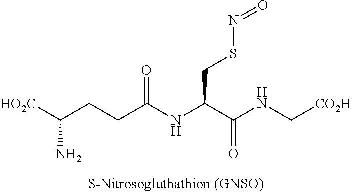

It has been shown that γ-Glu-X-Gly, where X represents an amino acid or an amino acid derivative; γ-Glu-Val-Y, where Y represents an amino acid or an amino acid derivative; γ-Glu-Ala; γ-Glu-Gly; γ-Glu-Cys; γ-Glu-Met; γ-Glu-Thr; γ-Glu-Val; γ-Glu-Orn; Asp-Gly; Cys-Gly; Cys-Met; Glu-Cys; Gly-Cys; Leu-Asp; γ-Glu-Met(O); γ-Glu-γ-Glu-Val; γ-Glu-Val-NH₂; γ-Glu-Val-ol; γ-Glu-Ser; γ-Glu-Tau; γ-Glu-Cys(S-Me)(O); γ-Glu-Leu; γ-Glu-Ile; γ-Glu-t-Leu; and γ-Glu-Cys(S-Me) activate a calcium receptor (WO 2007/055388 A1, WO 2007/055393 A1, WO 2008/139945 A1, WO 2008/139946 A1, and WO 2008/139947 A1). Furthermore, as illustrated in the examples, it has been found that a plurality of kinds of compounds having calcium receptor-activating activity, function to promote the secretion of GLP-1 or CCK. Thus, γ-Glu-X-Gly, where X represents an amino acid or an amino acid derivative; γ-Glu-Val-Y, where Y represents an amino acid or an amino acid derivative; γ-Glu-Ala; γ-Glu-Gly; γ-Glu-Cys; γ-Glu-Met; γ-Glu-Thr; γ-Glu-Val; γ-Glu-Orn; Asp-Gly; Cys-Gly; Cys-Met; Glu-Cys; Gly-Cys; Leu-Asp; γ-Glu-Met(O); γ-Glu-γ-Glu-Val; γ-Glu-Val-NH₂; γ-Glu-Val-ol; γ-Glu-Ser; γ-Glu-Tau; γ-Glu-Cys(S-Me)(O); γ-Glu-Leu; γ-Glu-Ile; γ-Glu-t-Leu; and γ-Glu-Cys(S-Me) may be used as a prophylactic or therapeutic composition for diabetes or obesity. The peptide or the low molecular weight compound can be used alone, or as a mixture of any two or more kinds thereof.

Of the above-mentioned compounds, exemplary compounds are γ-Glu-Val-Gly, γ-Glu-Cys-Gly, and cinacalcet. Additionally, γ-Glu-Cys is also preferable.

γ-Glu-Val-Gly, γ-Glu-Cys-Gly, and cinacalcet are particularly suitable for use as prophylactic or therapeutic compositions for obesity, and γ-Glu-Cys, γ-Glu-Cys-Gly, and cinacalcet are particularly suitable for use as prophylactic or therapeutic compositions for diabetes. The present invention includes the following examples of active ingredients in the prophylactic or therapeutic compositions indicated for diabetes or obesity:

prophylactic or therapeutic compositions for obesity comprising γ-Glu-Val-Gly as an active ingredient;

prophylactic or therapeutic compositions for obesity comprising γ-Glu-Cys-Gly as an active ingredient;

prophylactic or therapeutic compositions for obesity comprising cinacalcet as an active ingredient;

prophylactic or therapeutic compositions for diabetes comprising γ-Glu-Cys as an active ingredient;

prophylactic or therapeutic compositions for diabetes comprising γ-Glu-Cys-Gly as an active ingredient; and

prophylactic or therapeutic compositions for diabetes comprising cinacalcet as an active ingredient.

A commercially available product may be used as the above-mentioned peptide. Furthermore, the peptide may be obtained by appropriately employing a known technique such as a chemical synthesis method or an enzymatic synthesis method. The peptide can contain 2 to 3 amino acid residues, i.e., is relatively short, and hence, the chemical synthesis method is convenient. In the case of the chemical synthesis method, the oligopeptide may be synthesized or semi-synthesized by using a peptide synthesizer. An example of the chemical synthesis method includes a peptide solid phase synthesis method. The peptide synthesized as described above may be purified by general means such as ion exchange chromatography, reverse-phase high performance liquid chromatography, or affinity chromatography. These peptide solid phase synthesis methods and the subsequent peptide purification are well known in the technical field.

Furthermore, the peptide can also be produced by an enzymatic reaction. For example, the method described in WO 2004/011653 A1 can be used. That is, the peptide may also be produced by reacting one amino acid or dipeptide whose carboxyl terminus is esterified or amidated, and an amino acid having a free amino group (for example, an amino acid whose carboxyl group is protected) in the presence of a peptide-producing enzyme, and purifying the produced dipeptide or tripeptide. Examples of the peptide-producing enzyme include a culture of a microorganism having an ability of producing a peptide, microbial cells separated from the culture, or a processed product of the microbial cells, or a peptide-producing enzyme derived from the microorganism.

The peptide can not only be produced by such an enzymatic method or a chemical synthesis method as mentioned above, but also may exist, for example, in a plant such as a vegetable or a fruit, a microorganism such as a yeast, and a yeast extract. When the peptide exists in natural products, the peptide may be extracted from those natural products before use.

Furthermore, the peptide does not need to be isolated before use, and a fraction containing the peptide of the present invention in a large amount may be used. An example to be used is a yeast extract containing glutathione (γ-Glu-Cys-Gly) or a fraction thereof. The preparation of the yeast extract or the like may be conducted in the same manner as general yeast extract preparation. The yeast extract may be a treated product of yeast cells extracted with hot water, or may be a treated product of digested yeast cells. The fraction of the yeast extract is not particularly limited as long as the fraction contains glutathione. An example is a yeast extract containing γ-Glu-Cys or a fraction thereof. The preparation of the yeast extract or the like may be conducted in the same manner as general yeast extract preparation. The yeast extract may be a treated product of yeast cells extracted with hot water, or may be a treated product of digested yeast cells. The fraction of the yeast extract is not particularly limited as long as the fraction contains γ-Glu-Cys.

The peptide can also be in the form of a salt. When the peptide is in the form of a salt, the salt may be a pharmacologically acceptable salt. Examples of a salt with an acidic group such as a carboxyl group in the formula include, but are not limited to: an ammonium salt; a salt with an alkali metal such as sodium and potassium; a salt with an alkaline earth metal such as calcium and magnesium; an aluminum salt; a zinc salt; a salt with an organic amine such as triethylamine, ethanolamine, morpholine, pyrrolidine, piperidine, piperazine, and dicyclohexylamine; and a salt with a basic amino acid such as arginine and lysine. Examples of a salt with a basic group in a case where the basic group exists in the formula include, but are not limited to: a salt with an inorganic acid such as hydrochloric acid, sulfuric acid, phosphoric acid, nitric acid, and hydrobromic acid; a salt with an organic carboxylic acid such as acetic acid, citric acid, benzoic acid, maleic acid, fumaric acid, tartaric acid, succinic acid, tannic acid, butyric acid, hibenzoic acid, pamoic acid, enanthoic acid, decanoic acid, teoclic acid, salicylic acid, lactic acid, oxalic acid, mandelic acid, and malic acid; and a salt with an organic sulfonic acid such as methanesulfonic acid, benzenesulfonic acid, and p-toluenesulfonic acid.

Examples of the low molecular weight compound having calcium receptor activity include cinacalcet and analogous compounds of cinacalcet. Examples of the analogous compounds of cinacalcet include the compounds described in U.S. Pat. No. 6,211,244 A, U.S. Pat. No. 6,213,146 A, U.S. Pat. No. 5,688,938 A, U.S. Pat. No. 5,763,569 A, U.S. Pat. No. 5,858,684 A, U.S. Pat. No. 5,962,314 A, U.S. Pat. No. 6,001,884 A, U.S. Pat. No. 6,011,068 A, U.S. Pat. No. 6,031,003 A, WO 1995/011221 A1, WO 1996/012687 A1, WO 2002/059102 A1, and the like. As in the case of the above-mentioned peptide, the low molecular weight compound also includes that in the form of a salt.

<2>Prophylactic or Therapeutic Composition for Diabetes or Obesity

The peptide and the low molecular weight compound each having a calcium receptor-activating function may be used as active ingredients in the prophylactic or therapeutic composition for diabetes or obesity. In the present invention, the calcium receptor activator may be used alone, or may be used as a mixture of any two or more kinds thereof. Examples of the form of the prophylactic or therapeutic composition for diabetes or obesity include pharmaceuticals, quasi drugs, and foods.

A method of applying the prophylactic or therapeutic composition for diabetes or obesity is not particularly limited, and an oral administration, an invasive administration utilizing an injection or the like, a suppository administration, or a transdermal administration may be adopted. The prophylactic or therapeutic composition for diabetes or obesity may be administered in the form of a conventionally used pharmaceutical formulation by mixing its active ingredient with a solid or liquid non-toxic pharmaceutical carrier, which is suitable for an administration method such as an oral administration and an injection. An oral administration is one example. Examples of such a formulation include, but are not limited to: a form of a solid formulation such as a tablet, a granule, a powder, and a capsule; a form of a liquid formulation such as a solution, a suspension, and an emulsion; and a form of a lyophilizate or the like. Those formulations may be prepared by usual methods for preparing the formulations.

Examples of the above-mentioned non-toxic pharmaceutical carrier include, but are not limited to, glucose, lactose, sucrose, starch, mannitol, dextrin, fatty acid glyceride, polyethylene glycol, hydroxyethyl starch, ethylene glycol, polyoxyethylene sorbitan fatty acid ester, gelatin, albumin, amino acid, water, and physiological saline. Additionally, if required, a conventionally used additive agent such as a stabilising agent, a wetting agent, an emulsifier, a binder, and a tonicity agent may be added appropriately.

Furthermore, the prophylactic or therapeutic composition for diabetes or obesity may comprise, in addition to the peptide and/or the low molecular weight compound, one or more kinds of other calcium receptor activators.

Examples of the above-mentioned other calcium receptor activators include, but are not limited to, a cation such as a calcium cation and a gadolinium cation; a basic peptide such as polyarginine and polylysine; a polyamine such as putrescine, spermine, and spermidine; a protein such as protamine; and an amino acid such as phenylalanine. The calcium receptor activator may be used alone, or may be used as a mixture of any two or more kinds thereof. Of the above-mentioned other calcium receptor activators, a cation such as a calcium cation or a gadolinium cation can be used, and a calcium cation is a particular example. In other words, at least one kind of the other calcium receptor activators to be further added can be a cation.

When the above-mentioned other calcium receptor activators coexist with the peptide or the low molecular weight compound, stronger activation on a calcium receptor is observed. The ratio of the total of the peptide and the low molecular weight compound to the total of other calcium receptor activators in the prophylactic or therapeutic composition for diabetes or obesity is not particularly limited as long as a stronger activation on the calcium receptor is attained. For example, the mass ratio of the total of the other calcium receptor activators to the total of the peptide or the low molecular weight compound is preferably set to 1:100 to 100:1.

The dosage or intake of the prophylactic or therapeutic composition for diabetes or obesity may be any amount as long as the amount is effective for therapy or prophylaxis, and is appropriately adjusted depending on the age, gender, body weight, symptom, and the like of a patient. For example, in the case of an oral administration, the total amount of the peptide and the low molecular weight compound to be used can be 0.01 g to 10 g per kg body weight per one dose, and in another example, 0.1 g to 1 g per kg body weight per one dose. The administration frequency is not particularly limited, and the administration may be performed once to several times per day.

The content of the peptide or the low molecular weight compound in the prophylactic or therapeutic composition for diabetes or obesity is not particularly limited as long as the content is suited to the above-mentioned dosage. The content can be 0.000001% by mass to 99.9999% by mass, or in another example, 0.00001% by mass to 99.999% by mass, and in another example 0.0001% by mass to 99.99% by mass with respect to the dry weight.

The prophylactic or therapeutic composition for diabetes or obesity may also be used as a food having a therapeutic or prophylactic effect on diabetes or obesity, for example, a food in a container or a package indicating that the agent has a prophylactic or therapeutic effect on diabetes or obesity. The food includes the peptide or the low molecular weight compound in an amount of 0.000001% or more, 0.00001% or more, 0.00001% or more and 98% or less. The form of the food is not particularly limited, and the food may be produced in the same production method as a general food and with the same materials as those for the general food except that the peptide or the low molecular weight compound is blended. Examples of the food include, but are not limited to, a seasoning, a beverage, a health food, a processed agricultural product, a processed fishery product, and a processed animal product.

<3>Protamine

Protamine can be used as the calcium receptor activator as an active ingredient in the prophylactic or therapeutic composition for diabetes or obesity. The above detailed description on the case of the peptide or the low molecular weight compound is also basically applied mutatis mutandis to the case where the calcium receptor activator is protamine. An additional description is given below.

Protamine having a calcium receptor-activating function may be used as the active ingredient in the prophylactic or therapeutic composition for diabetes or obesity.

Protamine is particularly suitable for the prophylactic or therapeutic composition for diabetes. The present invention includes, for example, a prophylactic or therapeutic composition for diabetes comprising protamine as an active ingredient.

The prophylactic or therapeutic composition for diabetes or obesity may further comprise, in addition to protamine, one or more kinds of other calcium receptor activators.

When the above-mentioned other calcium receptor activators coexist with protamine, stronger activation on a calcium receptor is observed. The ratio of protamine to the total of other calcium receptor activators in the prophylactic or therapeutic composition for diabetes or obesity is not particularly limited as long as a stronger activation on the calcium receptor is possible. For example, the mass ratio of the total of the other calcium receptor activators to the total of protamine is preferably set to 1:100 to 100:1.

The dosage or intake of the prophylactic or therapeutic composition for diabetes or obesity may be any amount as long as the amount is effective for therapy or prophylaxis, and is appropriately adjusted depending on the age, gender, body weight, symptom, and the like of a patient. For example, in the case of an oral administration, the total amount of protamine can be 0.01 g to 10 g per kg body weight per dose and in another example 0.1 g to 1 g per kg body weight per dose. The administration frequency is not particularly limited, and the administration may be performed once to several times per day.

The content of protamine in the prophylactic or therapeutic composition for diabetes or obesity of the present invention is not particularly limited as long as the content is suited to the above-mentioned dosage. The content can be 0.000001% by mass to 99.9999% by mass, or in another example 0.00001% by mass to 99.999% by mass, and in another example 0.0001% by mass to 99.99% by mass with respect to the dry weight.

The prophylactic or therapeutic composition for diabetes or obesity may also be used as a food having a therapeutic or prophylactic effect on diabetes or obesity, for example, a food in a container or a package indicating that the agent has a prophylactic or therapeutic effect on diabetes or obesity. The food can contain protamine in an amount of 0.000001% or more, or in another example 0.00001% or more, or in another example 0.00001% or more and 98% or less. The form of the food is not particularly limited, and the food may be produced in the same production method as a general food and with the same materials as those for the general food except that protamine is blended. Examples of the food include, but are not limited to, a seasoning, a beverage, a health food, a processed agricultural product, a processed fishery product, and a processed animal product.

### EXAMPLES

Hereinafter, the present invention is more specifically described by way of examples. However, the scope of the present invention is not limited to these examples.

### Reference Example 1

### Synthesis of γ-Glu-Val-Gly

Boc-Val-OH (8.69 g, 40.0 mmol) and Gly-OBzl.HCl (8.07 g, 40.0 mmol) were dissolved in methylene chloride (100 ml) and the solution was kept at 0° C. Triethylamine (6.13 ml, 44.0 mmol), HOBt (1-hydroxybenzotriazole, 6.74 g, 44.0 mmol), and WSC.HCl (1-ethyl-3-(3-dimethylaminopropyl)carbodiimide hydrochloride, 8.44 g, 44.0 mmol) were added to the solution, and the mixture was stirred overnight at room temperature. The reaction solution was concentrated under reduced pressure, and the residue was dissolved in ethyl acetate (200 ml). The solution was washed with water (50 ml), a 5% citric acid aqueous solution (50 ml×twice), saturated brine (50 ml), a 5% sodium bicarbonate aqueous solution (50 ml×twice), and saturated brine (50 ml). The organic layer was dried over anhydrous magnesium sulfate, magnesium sulfate was removed by filtration, and the filtrate was concentrated under reduced pressure. The residue was recrystallized from ethyl acetate/n-hexane to obtain Boc-Val-Gly-OBzl (13.2 g, 36.2 mmol) as a white crystal.

Boc-Val-Gly-OBzl (5.47 g, 15.0 mmol) was added to a 4 N HCl/dioxane solution (40 ml), and the mixture was stirred at room temperature for 50 minutes. Dioxane was removed by concentration under reduced pressure, n-hexane (30 ml) was added to the residue, and the mixture was concentrated under reduced pressure. The procedure was repeated three times to quantitatively obtain H-Val-Gly-OBzl.HCl.

H-Val-Gly-OBzl.HCl and Z-Glu-OBzl (5.57 g, 15.0 mmol) described above were dissolved in methylene chloride (50 ml), and the solution was kept at 0° C. Triethylamine (2.30 ml, 16.5 mmol), HOBt (1-hydroxybenzotriazole, 2.53 g, 16.5 mmol), and WSC.HCl (1-ethyl-3-(3-dimethylaminopropyl)carbodiimide hydrochloride, 3.16 g, 16.5 mmol) were added to the solution, and the mixture was stirred at room temperature overnight for 2 days. The reaction solution was concentrated under reduced pressure, and the residue was dissolved in heated ethyl acetate (1500 ml). The solution was washed with water (200 ml), a 5% citric acid aqueous solution (200 ml×twice), saturated brine (150 ml), a 5% sodium bicarbonate aqueous solution (200 ml×twice), and saturated brine (150 ml). The organic layer was dried over anhydrous magnesium sulfate, magnesium sulfate was removed by filtration, and the filtrate was concentrated under reduced pressure. The precipitated crystal was collected by filtration and dried under reduced pressure to obtain Z-Glu(Val-Gly-OBzl)-OBzl (6.51 g, 10.5 mmol) as a white crystal.

Z-Glu(Val-Gly-OBzl)-OBzl described above (6.20 g, 10.03 mmol) was suspended in ethanol (200 ml), 10% palladium/carbon (1.50 g) was added to the suspension, and a reduction reaction was performed under a hydrogen atmosphere at 55° C. for 5 hours. During the reaction, 100 ml in a total volume of water were gradually added. The catalyst was removed by filtration using a Kiriyama funnel, and the filtrate was concentrated under reduced pressure to a half volume. The reaction solution was further filtered through a membrane filter, and the filtrate was concentrated under reduced pressure. The residue was dissolved in a small volume of water, and to the resultant, ethanol was added to precipitate a crystal, and the crystal was collected by filtration and dried under reduced pressure to obtain γ-Glu-Val-Gly as a white powder (2.85 g, 9.40 mmol).

ESI-MS: (M+H)⁺=304.1.

¹H-NMR (400 MHz, D₂O) δ (ppm): 0.87 (3H, d, J=6.8 Hz), 0.88 (3H, d, J=6.8 Hz), 1.99-2.09 (3H, m), 2.38-2.51 (2H, m) 3.72 (1H, t, J=6.35 Hz), 3.86 (1H, d, J=17.8 Hz), 3.80 (1H, d, J=17.8 Hz), 4.07 (1H, d, J=6.8 Hz).

### Example 1

### Function to Promote Secretion of CCK

A CCK-producing cell strain STC-1 derived from the mouse small intestine was cultured in a Dulbecco's modified Eagle's medium supplemented with 10% FBS at 37° C. in the presence of 5% CO₂. The STC-1 cells were cultured in a 48-well plate for 2 to 3 days until the cells reached a subconfluent state. Prior to the addition of each of the samples, the wells were washed with a Hepes B buffer (140 mM NaCl, 4.5 mM KCl, 20 mM Hepes, 1.2 mM CaCl₂, 1.2 mM MgCl₂, 10 mM D-glucose, 0.1% BSA, pH 7.4), 100 of a sample solution obtained by dissolving each of the samples in the same buffer were added to the wells, and incubation was performed at 37° C. for 60 minutes. γ-Glu-Cys-Gly (Sigma-Aldrich Japan K.K.), γ-Glu-Val-Gly, and cinacalcet were used as the samples. Further, the Hepes B buffer was used as a control. After supernatant collection, the cells were precipitated by centrifugation (800×g, 5 minutes, 4° C.), and 80 μl of the supernatant were collected and cryopreserved.

After the supernatant had been pretreated with an OASIS cartridge (C18), the concentration of CCK in the supernatant was measured with an Enzyme immuno assay kit (Phoenix Pharmaceuticals).

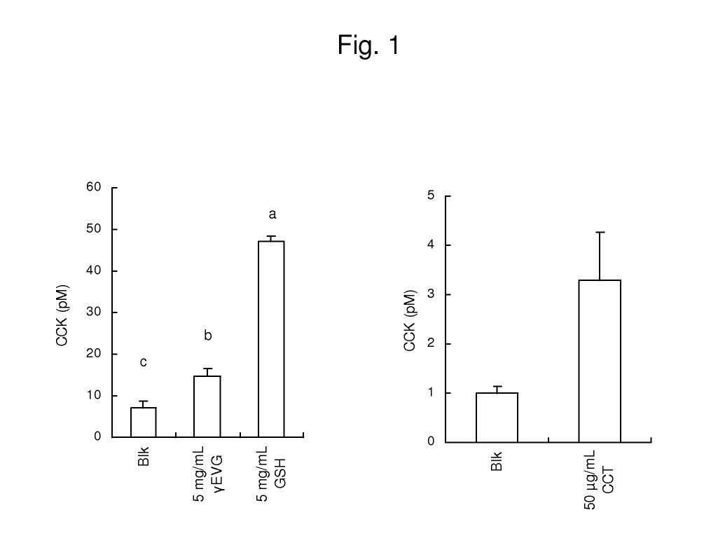

FIG. 1 illustrates the results. The results showed that each of γ-Glu-Cys-Gly, γ-Glu-Val-Gly, and cinacalcet functioned to promote the secretion of CCK.

### Example 2

### Function to Promote Secretion of GLP-1

A GLP-1-producing cell strain GLUTag derived from the mouse large intestine was cultured in a Dulbecco's modified Eagle's medium supplemented with 10% FBS at 37° C. in the presence of 5% CO₂. The GLUTag cells were cultured in a 48-well plate for 2 to 3 days until the cells reached a subconfluent state. Prior to the addition of each of samples, the wells were washed with a Hepes B buffer (140 mM NaCl, 4.5 mM KCl, 20 mM Hepes, 1.2 mM CaCl₂, 1.2 mM MgCl₂, 10 mM D-glucose, 0.1% BSA, pH 7.4), 80 μl of a sample solution obtained by dissolving each of the samples in the same buffer were added to the wells, and incubation was performed at 37° C. for 60 minutes. γ-Glu-Cys-Gly and cinacalcet were used as the samples. Further, the Hepes B buffer was used as a control. After supernatant collection, the cells were precipitated by centrifugation (800×g, 5 minutes, 4° C.), and 70 μl of the supernatant were collected and cryopreserved. The concentration of GLP-1 in the supernatant was measured with a commercially available Enzyme immuno assay kit (Yanaihara Institute Inc.).

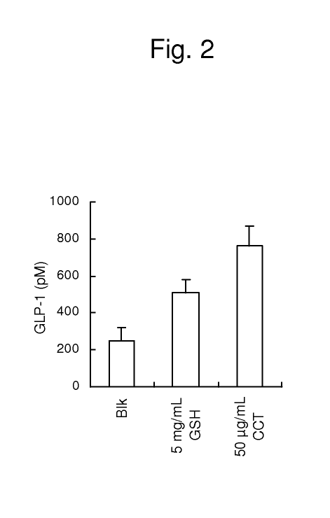

FIG. 2 illustrates the results. The results showed that each of γ-Glu-Cys-Gly and cinacalcet functioned to promote the secretion of GLP-1.

### Example 3

### Function to Promote Secretion of GLP-1 (2)

In the same manner as in Example 2, a function to promote the secretion of GLP-1 was examined using a GLP-1-producing cell strain GLUTag. γ-Glu-Cys and protamine were used as samples.

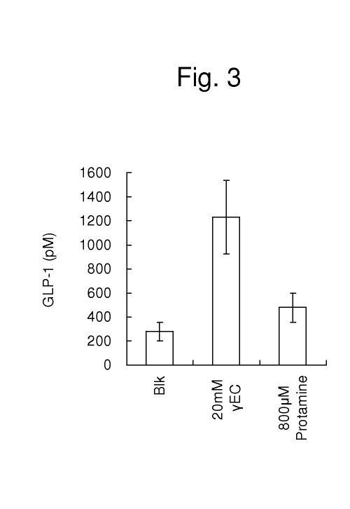

FIG. 3 illustrates the results. The results showed that each of γ-Glu-Cys and protamine functioned to promote the secretion of GLP-1.

### Example 4

### GLP-1 Secretion Test in Rats

After male SD rats (8-week-old) had been preliminarily fed, fasted overnight, and subjected to celiotomy under ketamine/xylazine anesthesia. GLP-1-producing cells are mainly distributed in an ileal site. Thus, after a ligated ileal loop (30 cm) had been prepared, a catheter was placed into the ileal mesenteric vein, and blood was collected from the catheter at the time of 0 minute (0 time). A sample (2 mL of deionized water, 20 mg of γ-Glu-Cys/2 mL of deionized water) was administered into the ligated ileum loop, blood was collected at 30, 60, 90, and 120 minutes after administration, respectively, and the plasma GLP-1 concentration was measured with an Enzyme immuno assay kit (manufactured by Yanaihara Institute Inc.).

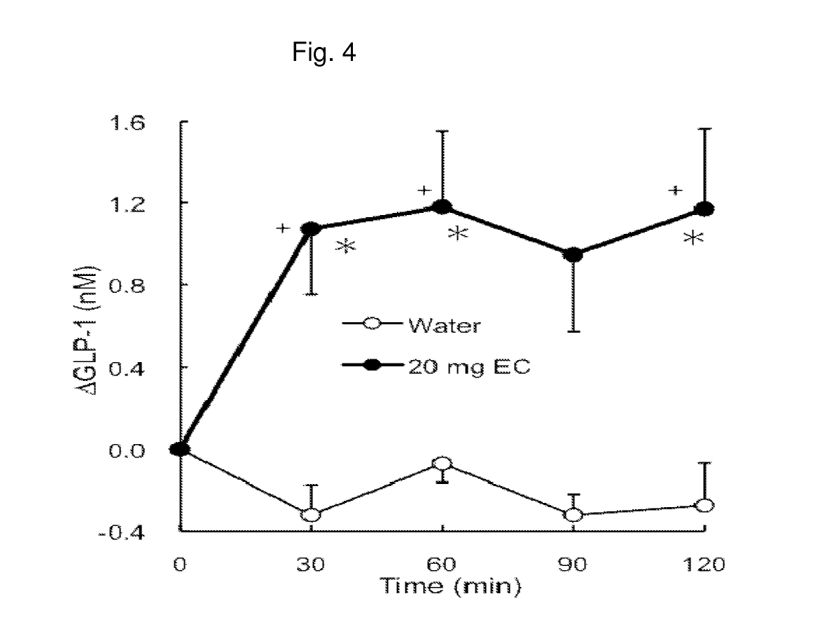

FIG. 4 illustrates the results. Rats to which γ-Glu-Cys was administered showed a significantly higher elevation of plasma GLP-1 concentration as compared to a water administration group. Therefore, it was confirmed that γ-Glu-Cys functioned to promote the secretion of GLP-1 in rats.

### Example 5

### Glucose Tolerance Test in Rats

After male SD rats (7-week-old) had been preliminarily fed (for 3 to 4 days), and fasted overnight, they were subjected to an oral glucose tolerance test. Tail vein blood before specimen administration was collected, and a sample [glucose solution alone (2 g/kg weight)/glucose solution comprising γ-Glu-Cys (160 mg/kg weight)] was orally administered with a feeding tube. After sample administration, tail vein blood was collected at 15, 30, 60, 90, and 120 minutes, and the plasma glucose concentration was measured with a commercially available kit (manufactured by Wako Pure Chemical Industries, Ltd.).

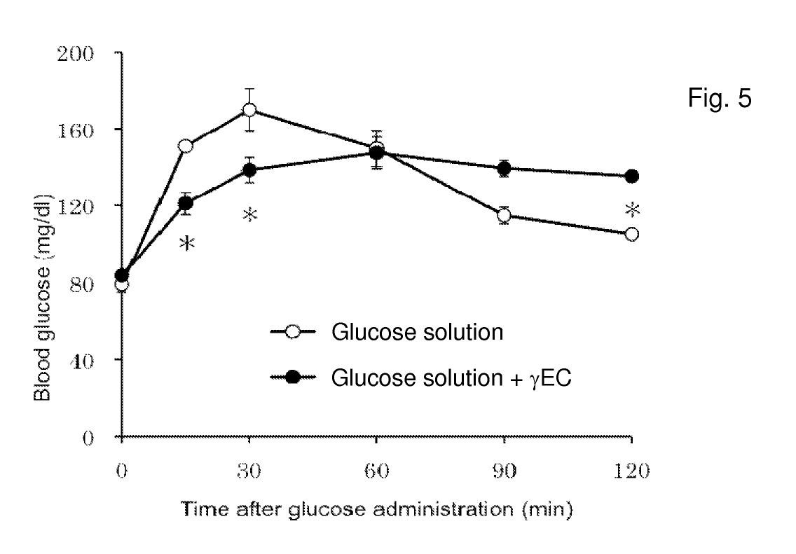

FIG. 5 illustrates the results. At 15 and 30 minutes after glucose administration, rats to which γ-Glu-Cys was administered showed a significantly lower blood glucose concentration value as compared to a group to which a glucose solution was administered alone. Therefore, it was found that γ-Glu-Cys functioned to suppress the elevation of plasma glucose. It is understood that the calcium receptor activator having an activity of promoting the secretion of GLP-1 functions to suppress the elevation of blood sugar.

### INDUSTRIAL APPLICABILITY

The present invention provides a prophylactic or therapeutic composition for diabetes or obesity, which is highly safe to the living body.

While the invention has been described in detail with reference to preferred embodiments thereof, it will be apparent to one skilled in the art that various changes can be made, and equivalents employed, without departing from the scope of the invention. Each of the aforementioned documents is incorporated by reference herein in its entirety.

What is claimed is:

1. A method for prophylaxis or treatment of diabetes or obesity, comprising administering a composition comprising a calcium receptor activator to a subject in need of prophylaxis or treatment of diabetes or obesity.

2. The method according to claim 1, wherein the calcium receptor activator is selected from the group consisting of γ-Glu-X-Gly, where X represents an amino acid or an amino acid derivative except for cysteine; γ-Glu-Val-Y, where Y represents an amino acid or an amino acid derivative; γ-Glu-Ala; γ-Glu-Gly; γ-Glu-Cys; γ-Glu-Met; γ-Glu-Thr; γ-Glu-Val; γ-Glu-Orn; Asp-Gly; Cys-Gly; Cys-Met; Glu-Cys; Gly-Cys; Leu-Asp; γ-Glu-Met(O); γ-Glu-γ-Glu-Val; γ-Glu-Val-NH₂; γ-Glu-Val-ol; γ-Glu-Ser; γ-Glu-Tau; γ-Glu-Cys(S-Me)(O); γ-Glu-Leu; γ-Glu-t-Leu; γ-Glu-Cys(S-Me); cinacalcet; an analogous compound of cinacalcet; and combinations thereof.

3. The method according to claim 2, wherein X is selected from the group consisting of Cys(SNO), Cys(S-allyl), Gly, Cys(S-Me), Abu, and Ser; and Y is selected from the group consisting of Gly, Val, Glu, Lys, Phe, Ser, Pro, Arg, Asp, Met, Thr, His, Orn, Asn, Cys, and Gln.

4. The method according to claim 2, wherein the calcium receptor activator is γ-Glu-Val-Gly or cinacalcet.

5. The method according to claim 2, wherein the calcium receptor activator is γ-Glu-Cys.

6. The method according to claim 1, wherein the calcium receptor activator is protamine.

7. The method according to claim 1, wherein the composition comprises γ-Glu-Cys in an amount of 0.000001% or more.

8. The method according to claim 1, wherein the composition comprises protamine in an amount of 0.000001% or more.
